(19) United States
(12) Patent Application Publication
(10) Pub. No.: US 2015/0273235 A1
(43) Pub. Date: Oct. 1, 2015

(54) DEVICES AND METHOD OF CAUSING CHEMICAL REACTION TO SUPPLEMENT VITAMIN D PRODUCTION

(71) Applicant: Once Innovations, Inc., Plymouth, MN (US)

(72) Inventor: Zdenko Grajcar, Orono, MN (US)

(21) Appl. No.: 14/670,653

(22) Filed: Mar. 27, 2015

Related U.S. Application Data
(60) Provisional application No. 61/971,584, filed on Mar. 28, 2014.

(51) Int. Cl. A61N 5/06; A01G 7/04; H05B 33/08; A01G 1/00

(52) U.S. Cl. CPC A61N 5/0616; A01G 1/001; A01G 7/045; H05B 33/0842; A61N 2005/0626; A61N 2005/0652; A61N 2005/0661

(57) ABSTRACT

A method of supplementing vitamin D in a living organism by using an artificial lighting system. The lighting system includes a plurality of lighting devices that emit light at a predetermined wavelength and intensity known to cause the synthesis of vitamin D by the living organism. A controller is provided that controls both the intensity and wavelength of light so that the system can be used for different living organisms. The plurality of lighting devices also pulse, or continuously provide periods of light followed immediately by periods of no light where the periods of light and no light depend on the energy needed for the living organism to synthesize vitamin D and the time needed for the synthesis to occur before additional energy is accepted by the living organism in a manner that synthesizes additional vitamin D.

### CLAIM OF PRIORITY

This patent application claims the benefit of U.S. Provisional Patent Application Ser. No. 61/971,584, filed Mar. 28, 2014, entitled “Devices and Method of Causing Chemical Reaction to Supplement Vitamin D Production,” which is incorporated by reference herein in full.

### BACKGROUND

This invention relates to chemical reactions. More specifically this invention relates to a device and method that causes a person to produce more vitamin D.

A problem exists in the art regarding the supplementation of vitamin D for humans. In particular, recent studies have shown that vitamin D supplements have no known health benefits for humans. Previously tests had linked vitamin D production by an individual to reducing risk of heart attack, stroke, cancer and other ailments, yet when provided with vitamin D supplements test subjects showed no such positive effect. Some scientists theorize that vitamin D does in fact reduce risk of heart attack, stroke, cancer and other ailments; however, the supplements that are being ingested are ineffective at raising vitamin D levels of individuals. Specifically a test has shown that ingested vitamin D supplements are not effective at raising the level of vitamin D to above 21 nanograms per milliliter in the blood of a person.

It is well known in the art that light is responsible for causing certain photo-chemical reactions. As one example, when an individual is exposed to sunlight a photochemical reaction occurs to produce vitamin D. In particular the body synthesizes vitamin D (specifically cholecalciferol) in the skin, from cholesterol, in response to doses of specific UV (ultra violet) light provided by the sun. However, the skin can only handle so much UV light and once a certain dose of UV light is received the additional light provided is wasted and instead can be harmful, resulting in sunburn.

Mushrooms similarly have been shown to produce vitamin D when periodically exposed to UV lighting. Specifically, the exposure of merely five minutes of UV light has been shown to greatly increase the amount of vitamin D2 within a mushroom.

Similarly, during the photosynthesis process plants absorb different frequencies of light to cause photosynthesis to occur. In particular photosynthetically active radiation (PAR) is radiation in the spectral range from approximately 400 nanometers (nm) to 700 nm. Also known in the art is that chlorophyll, the most abundant plant pigment and the pigment responsible for plant metabolism is most efficient at capturing red and blue light. During photosynthesis the chlorophyll pigments in a plant absorb photons in order to drive a metabolic process and dissipate other energy within the photons. Simultaneously other pigments that are red/farred and blue/UV-A and UV-B photosensors or photoreceptors chemically react to adjust the behavior and development of the plant. Thus, by providing red and blue spectrum light, plants have been shown to grow at increased rates.

In addition, plants also need turn over, or time in the dark. In particular, when a pigment has accepted a photon and is going through the metabolic process, the pigment cannot accept additional photons. Still, when additional photons bombard the plant the pigments will continue to attempt to metabolize thus straining or fatiguing the plant. Thus dark time is needed to allow the pigments to complete the metabolic process and to restart the process. Thus, just as humans need sleep, plants similarly need down time to optimize the metabolic process.

In particular as chemical reactions occur an electron transport chain (ETC) is formed transferring electrons to cause the chemical reaction. In plants this is referred to as a photosynthetic electron transport chain or PETC. In the case of vitamin D synthesis, to synthesize vitamin D a predetermined amount of time is required for the electron transfer chain to transfer the quantum of energy provided by light or radiation at a predetermined wavelength and intensity to cause the resulting chemical reaction, in this instance the synthesis of vitamin D. Additional energy provided during this time is not useful to the vitamin D synthesis and instead merely produces additional energy that must be accounted for by the living organism, either through protective chemical reactions or through destructive reactions. As discussed, in humans the additional intake of light by the skin results in a sunburn.

Thus, for the photo-chemical reaction associated with vitamin D synthesis only a certain dose of light, or maximum exothermal dose (MED) is required to cause the synthesis, similar to only a maximum MED is required for photosynthesis to occur. The additional light is thus wasted and again can be harmful to the living organism.

Therefore, a principle object of the present invention is to cause, control and enhance chemical reactions using a light power source.

Yet another object of the present invention is to provide a system and method to supplement vitamin D production of an individual.

These and other objects, features and advantages will become apparent from the rest of the specification.

### Overview

A system and method of supplementing vitamin D in a living organism, including plants and animals. A lighting system is provided that can be controlled by a controller. The system has a plurality of lighting devices that are designed to periodically emit light at wavelengths, intensities and for periods of time required to provide sufficient energy to cause the synthesis of vitamin D by the living organism. The system then no longer provides light while the chemical reaction of the synthesis is taking place to ensure excess radiation that can be harmful to the living organism is eliminated. Light is then provided again at the wavelength, intensity and period when the living organism is ready to synthesize more energy into vitamin D without harmful effects on the living organism.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIG. 1 is a perspective view of a lighting device.

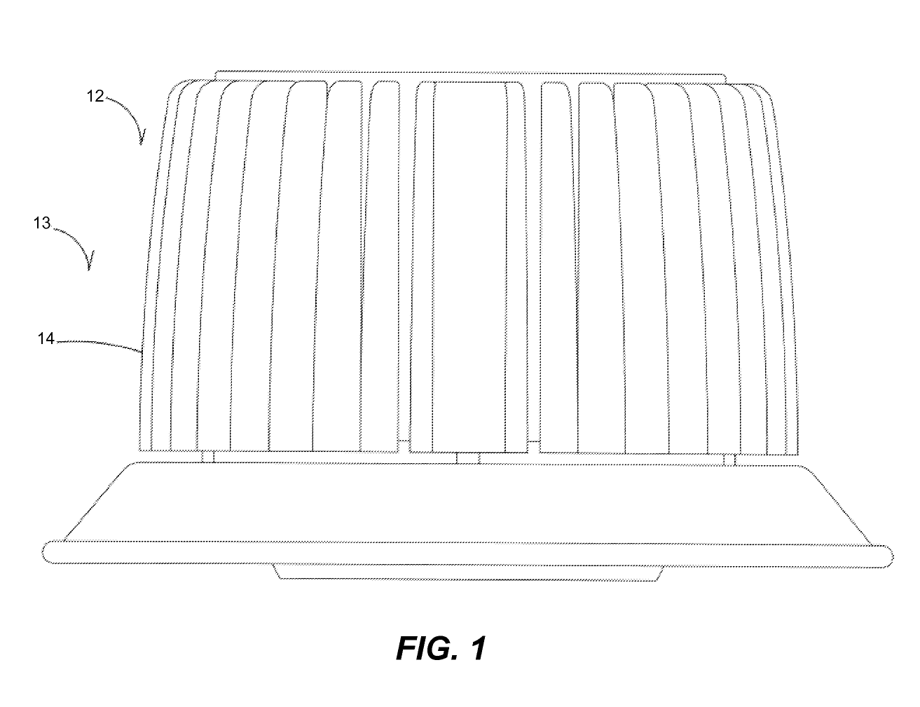

FIG. 2 is a side plan view of a lighting device.

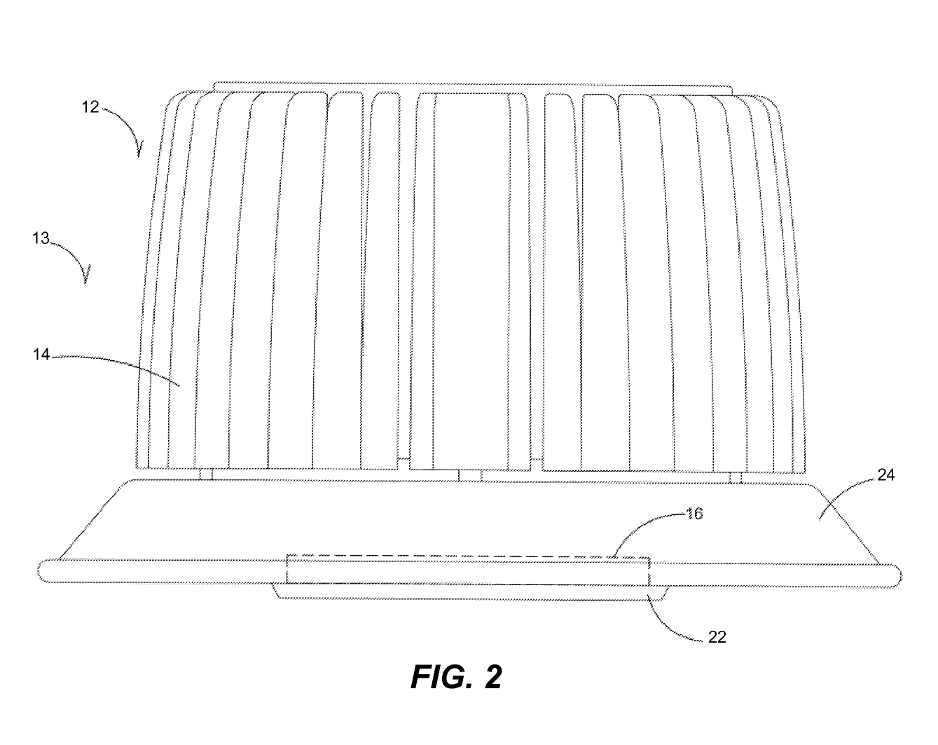

FIG. 3 is a top plan view of a circuit board with circuitry for a lighting device.

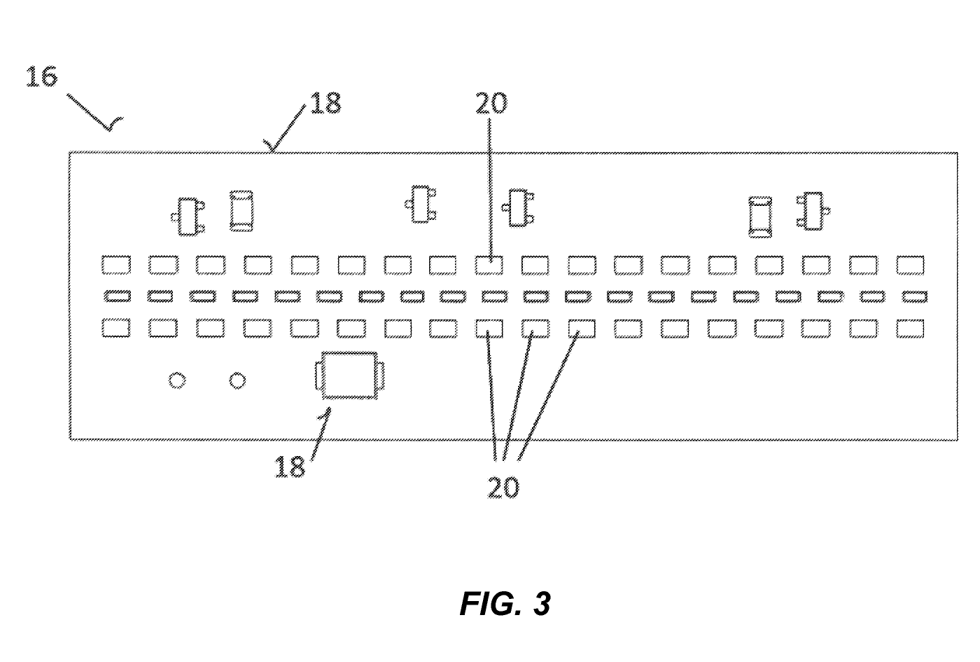

FIG. 4 is a schematic of a controller used with a lighting device.

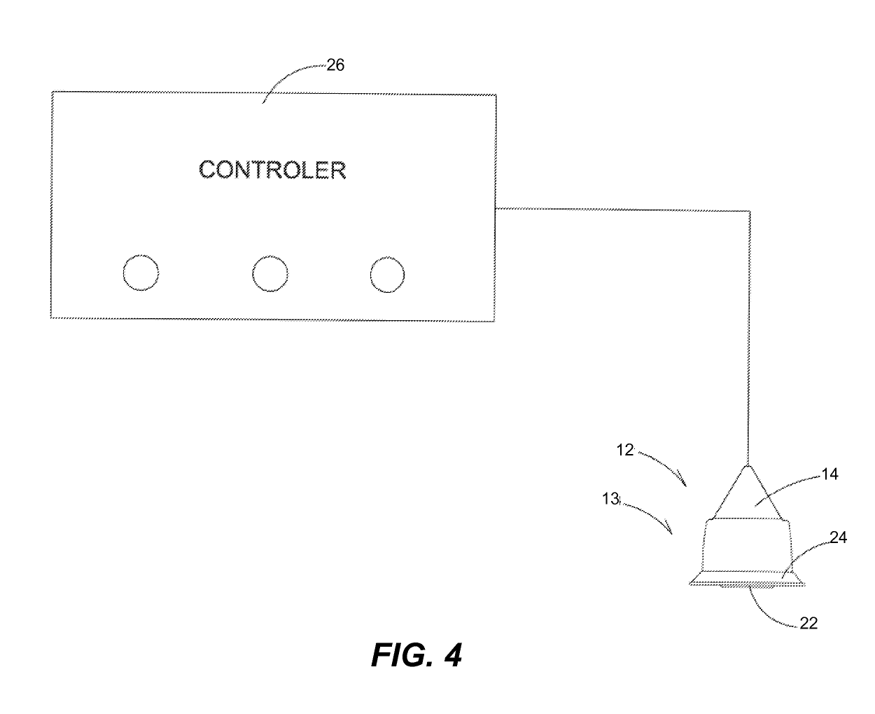

FIG. 5 is a side plan view of a lighting system in use by a person.

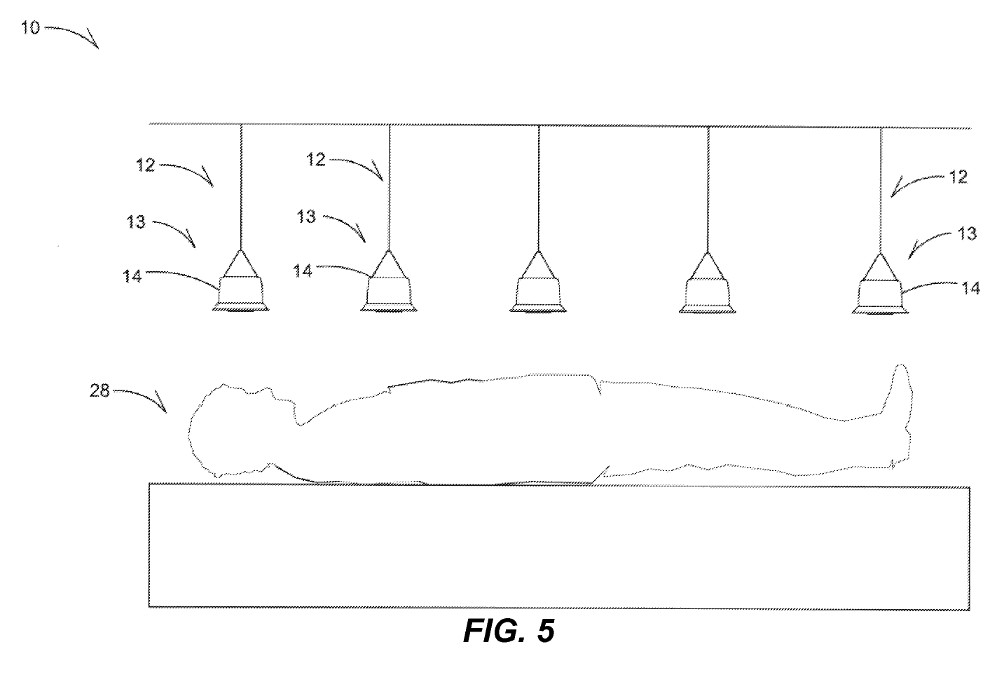

### DETAILED DESCRIPTION

The Figures show a system 10 that includes a photon producing source 12. The photon producing source 12 can be any device that emits photons, whether a lamp or lighting device that emits light in the visible spectrum, whether X-rays, ultraviolet light, infarred light, light from 300 nm to 750 nm. Preferably the photon producing source 12 is able to provide light in a narrow range of wavelengths. The photon producing source 12 can be an incandescent light, compact florescent light, high pressure sodium lamp, LED lighting device or the like without falling outside the scope of this disclosure.

As an example only, in one embodiment the photon producing source 12 is a light emitting device 13 that has a housing 14, surrounding a printed circuit board (PCB) 16 having driving circuitry 18 and a plurality of light emitting diodes (LEDs) 20 thereon that each emit a light of predetermined wavelength to provide a lighting output. The housing 14 can also comprise a lens element 22 and shade element 24 for diffusing the light. The photon producing source 12 of this embodiment can be electrically and operably connected to a controller 26 such as a dimming device that can control the voltage or current supplied to the photon producing source 12 and/or light emitting device 13 and thus control either the intensity of light output or wavelength of light emitted. Several examples of embodiments of these light emitting device are presented in U.S. Pat. Pub. No. 2011/0101883 to Grajcar; U.S. Pat. Pub. No. 2011/0109244 to Grajcar; U.S. Pat. Pub. No. 2011/0210678 to Grajcar; U.S. Pat. Pub. No. 2011/0228515 to Grajcar; U.S. Pat. Pub. No. 2011/0241559 to Grajcar; U.S. Pat. Pub. No. 2011/0273098 to Grajcar; U.S. patent application Ser. No. 13/452,332 to Grajcar; and/or U.S. Pat. Prov. Appl. No. 61/570,552 to Grajcar. Each of these references are incorporated in full into this specification.

In one embodiment as shown in the Figures the system 10 has a plurality of light emitting devices 13 arranged in a side by side configuration to emit light over a pre-determined area. In particular the pre-determined area in one embodiment is the area covered by a person 28 who is receiving a treatment of light. Specifically determined is the optimum dose of light or radiation in order to cause the chemical reaction within the body to cause vitamin D production. Thus, depending upon the dose required, whether a certain intensity or time is required is predetermined to determine how to maximize the chemical reaction that forms the vitamin D but prevents burning of the skin. Specifically, also predetermined is the amount of time required for the chemical reaction to complete itself once the optimum amount of energy is provided to cause the chemical reaction. During this period the lighting devices are programmed to not emit additional light that can no longer be accepted by a person's skin and causes burning.

In some embodiments the period of time between the end of the presentation of dose and for the chemical reaction to complete, such that the skin is again able to accept another dose of the energy or light, can be five miliseconds (5 ms) or less. In these embodiments the lighting devices are created or programmed to quickly turn off and on and such operation is perceived by an individual as a flicker of the lighting device 13. In other embodiments the frequency of the on and off function of the lighting devices 13 is so frequent that it is not perceivable to an individual, but is detected by the cells responsible for the chemical reaction to create the vitamin D. Alternatively the photoperiod is greater than 5 ms, even up to thirty minutes.

In operation a dose of light is predetermined for an individual. The dose light represents a predetermined amount of energy provided by light depending upon the characteristics of light including but not limited to the intensity, wavelength and duration of the light. Thus in one embodiment, for example only, a UV wavelength range light (100 nm-400 nm) at two (2) footcandles for ten (10) miliseconds may provide that exact dose of energy to cause a certain person's cells to synthesize the energy and create vitamin D. Also predetermined is the amount of down time or darkness required to complete the chemical reaction before additional energy should be provided to cause the chemical reaction to again occur. In this manner, additional harmful energy from the light that can result in burning or harm to cells or the skin is eliminated.

Once the characteristics of the light and predetermined dark period to allow the completion of the chemical reaction are determined the lighting devices are created and/or programmed to provide light having the predetermined characteristics that can be modulated to provide the exact dose and darkness periods required to cause the desired chemical reaction and allow the reaction to be completed so that the cells are again ready to receive a next dose of energy from the lighting devices 13. In this manner vitamin D can be synthesized by the dose of light provided by the lighting devices 13 thus increasing or supplementing vitamin D production for an individual without damaging cells or causing sunburn or other negative effects often associated with UV or other light.

As a result of allowing the chemical reaction to complete and allowing time for the body or plant to have down time to prepare to be ready for another dose for an additional chemical reaction, the additional unneeded radiation that causes a sunburn or provides additional energy for destructive or protective chemical reactions of the living organism is minimized or eliminated. Therefore less overall energy is required by the living organism and the living organism is able to get back to a state where synthesis of vitamin D is possible sooner than without the time of no light such that the vitamin D synthesis is not only more efficient than constant light or radiation but additionally significantly more vitamin D is synthesized as a result of allowing the chemical reaction to occur over and over again without impedance by additional protective chemical reactions. Consequently by pulsing or providing intermittent periods of no light vitamin D production is supplemented or enhanced compared to vitamin D produced when light or radiation is constant, the effect of which is maximized as a result of predetermining the energy needed and time required for the synthesis to occur.

In addition, because the triggering of the chemical reaction is a natural chemical response of an individual's body, unlike vitamin D supplements that are ingested, yet fail to increase the level of vitamin D detectable in the blood of a person in the body, the light treatment from the system effectively increases these vitamin D levels and thus provides a supplement for the vitamin D level of an individual. Therefore all of the stated problems are addressed and overcome.

A number of implementations have been described. Nevertheless, it will be understood that various modification may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed:

1. A lighting system for enhancing vitamin D synthesis of a living organism comprising:
at least one light emitting device that emits light at a predetermined wavelength and intensity that causes vitamin D to be synthesized by a living organism that receives the emitted light;
said at least one light emitting device during operation continuously alternating between periods of emitting light and periods of not emitting light;
wherein the length of time of the periods of not emitting light coincides with the amount of time required for the living organism to synthesize vitamin D.

2. The lighting system of claim 1 wherein the period of not emitting light is less than 10 ms.

3. The lighting system of claim 1 wherein the light emitting device comprises light emitting diodes.

4. The lighting system of claim 1 further comprising a controller electrically connected to the lighting device to determine the wavelength and intensity of the at least on light emitting device.

5. The lighting system of claim 1 wherein the living organism is a human.

6. The lighting system of claim 1 wherein the living organism is a plant.

7. The lighting system of claim 1 wherein the predetermined wavelength is between 100 nm and 400 nm.

8. A method of supplementing vitamin D in a living organism, steps comprising:
providing a system having at least one light emitting device;
emitting light with the at least one light emitting device having a wavelength between 100 nm and 400 nm onto the living organism;
emitting light with the at least one light emitting device during a predetermined time period;
during the predetermined time period continuously alternating between periods of less than 10 ms when light is emitted and periods of less than 10 ms when no light is emitted to cause the living organism to produce vitamin D during the predetermined time period.

9. The method of claim 8 wherein the predetermined period is 30 minutes.

10. The method of claim 8 wherein the period when light is emitted is the same as the period when no light is emitted.

11. The method of claim 8 wherein the period when light is emitted is different than the period when no light is emitted.

12. The method of claim 8 wherein the living organism is an animal.

13. The method of claim 8 wherein the living organism is a plant.

14. The method of claim 8 wherein the period of time when no light is emitted coincides with the length of time of the chemical reaction of cells of the living organism to synthesize the energy from the light and create vitamin D.

15. The method of claim 8 wherein the light is emitted at at least 2 footcandles.

16. The method of claim 8 wherein the light is emitted at less than 2 footcandles.
